(19) United States
(12) Patent Application Publication
(10) Pub. No.: US 2025/0186801 A1
(43) Pub. Date: Jun. 12, 2025

(54) SYSTEMS AND METHODS FOR PERTURBATION USING PLURAL NON-ZERO ORBITAL ANGULAR MOMENTUM (OAM) QUANTUM PARTICLES

(71) Applicant: University of Maryland, College Park, College Park, MD (US)

(72) Inventors: Wolfgang Losert, Bethesda, MD (US); Patrick Kanold, Bethesda, MD (US); Samira Sadat Aghayee, Rockville, MD (US); Aminah Sheikh, Gaithersburg, MD (US); Aleksandr V. Smirnov, Gainesville, VA (US)

(21) Appl. No.: 18/971,597

(22) Filed: Dec. 6, 2024

Related U.S. Application Data
(60) Provisional application No. 63/607,139, filed on Dec. 7, 2023.

(51) Int. Cl. A61B 6/42; A61B 6/40

(52) U.S. Cl. CPC A61N 5/0622; A61B 5/0071; G02B 27/0927; G01N 33/4833

(57) ABSTRACT

Systems and methods of the present disclosure include plural non-zero Orbital Angular Momentum (OAM) quantum particles utilized to generate a toroidal (and, in aspects super-twised) intensity of Electro-Magnetic (EM) field radiation that is delocalized in space and spans the whole toroid with zero or near-zero amplitude at its center are tuned (e.g., varied) in diameter to enable perturbation, e.g., stimulation, of a target at a target plane (or planes) with, for example, effective depolarization and activation of the target while inhibiting the establishment of spurious and damaging focal regions of the quantum particles' intensity.

### CROSS-REFERENCE TO RELATED APPLICATIONS

This application claims the benefit of, and priority to, U.S. Provisional Patent Application No. 63/607,139, filed on Dec. 7, 2023, the entire contents of which are hereby incorporated herein by reference.

### STATEMENT REGARDING FEDERALLY SPONSORED RESEARCH/DEVELOPMENT

This invention was made with government support under U19 NS107464 awarded by the National Institutes of Health, The government has certain rights in the invention.

### FIELD

The present disclosure relates to perturbation and, more specifically, to systems and methods for perturbation using plural non-zero orbital angular momentum (OAM) quantum particles.

### BACKGROUND

Photoperturbation has emerged as a powerful technique for investigating live cell functional dynamics, e.g., neuronal circuitry in living subjects. For example, photostimulation of multiple targeted neurons at the same time with single-cell resolution and simultaneous optical readout of neuronal activity provides the ability to understand brain function, since neuronally encoded information can be represented by groups of interacting neurons that fire within short time intervals from each other. As another example, photostimulation provides the ability to trigger firing of neurons and other cells and/or cellular components in the absence of a sensory stimulus.

### SUMMARY

Although perturbation of living subjects is generally known, perturbation of cells and/or cellular components such as neurons in living subjects remains a challenge due to the need to achieve sufficient sensitivity, perturb at a targeted plane, and minimize or inhibit tissue damage.

The present disclosure provides systems and methods for perturbation, e.g., of microscopic objects such as cells, cellular components, viruses, etc., using plural non-zero orbital angular momentum (OAM) quantum particles. The aspects and features of the present disclosure, as detailed below, provide sufficient sensitivity to enable this perturbation or manipulation while minimizing or inhibiting perturbation outside of the targeted plane (which may be at tissue depths of, in aspects, up to 2 or 3 mm), and minimizing or inhibiting tissue damage.

The present disclosure, more specifically, utilizes multiple quantum particles, e.g., photons, with non-zero OAM. Non-zero OAM quantum particles are in a distinct topological quantum state that is spatially extended in its localization probability, thus generating a toroidal intensity distribution field (waveform) that is delocalized in space and spans the whole toroid with zero amplitude at its center. Accordingly, by tuning the diameter of the toroidal intensity field produced by the quantum non-zero OAM quantum particles (e.g., to substantially overlap, for example, a neuronal soma membrane with the center approximated over the nuclear region of the neuron), perturbation at the target plane (or planes) is achieved with effective membrane depolarization and activation of the neuron at the target plane (or planes) while inhibiting the establishment of spurious and tissue-damaging focal regions outside of the target plane as well as in the nuclear region of the neuron, which is most vulnerable to damage. Where a large number of neurons are to be perturbed, the diameter of the toroidal intensity field produced by the non-zero OAM quantum particles can be tuned for each neuron or, alternatively, can be tuned to an average soma diameter (or other suitable approximation of its outer membrane diameter) for one or more groups of neurons to achieve a similar result while reducing the number of individual tuning adjustments required.

Provided in accordance with aspects of the present disclosure is a system including a laser or other suitable coherent source of particles configured to generate a plurality of particles, a plurality of components, e.g., optical components, configured to convert the plurality of particles into non-zero orbital angular momentum (OAM) quantum particles defining an wavefront phase vortex or other suitable wavefront configuration, and an focus or objective (or other suitable component) configured to condense the vortex to a target plane within a depth of tissue, such as live tissue.

In an aspect of the present disclosure, the vortex is an optical wavefront phase vortex configured to achieve photoperturbation while minimizing tissue damage.

In an aspect of the present disclosure, the system further includes a mode selector configured to select a mode of the vortex to adjust a diameter (or other parameter(s)) of the vortex, e.g., super-twisting, at the target plane. Spurious perturbations are avoided or limited by adjusting the diameter of the vortex to a target within the target plane, e.g., a targeted cell soma membrane within the target plane. In such aspects, the mode selector may be configured to adjust the diameter (or other parameter(s)) of the vortex at the target plane to correspond to a size of a neuronal soma (or other target cell or cell component).

In another aspect of the present disclosure, the system further includes an adjustment mechanism e.g., diffraction grating, a galvanometric mirror, or an acousto-optics deflector, configured to adjust a location of the vortex within the target plane. The adjustment mechanism, in such aspects, may be configured to adjust the location of the wavefront vortex along the target plane to correspond to a location of a neuron (or other target cell or cell component).

In still another aspect of the present disclosure, the system further includes a multiplier mechanism configured to generate multiple vortices at the target plane or multiple target planes. In such aspects, each vortex may correspond to a different neuron (or other target cell or cell component).

In yet another aspect of the present disclosure, the plurality of components includes a plurality of quantum particle manipulation components. Such components may include, for example, one or more optical components such as a physical or virtual lens configuration. A Spatial Light Modulator (SLM), acousto-optical, and/or a Pockels Cell system may also be utilized. Other suitable devices for this purpose include deformable mirrors, adaptive (tunable) lenses, segmented waveplates, liquid crystal retarders, holographic diffractive optical elements, GRIN plates, and additively manufactured, photopolymerized, ablated or etched out surfaces.

In still yet another aspect of the present disclosure, each quantum particle of the plurality of quantum particles is a photon. The plurality of photons may include, in aspects, two photons. In aspects where the wavefront phase vortex is an optical vortex, the optical vortex may an optical vortex of super-twisted light.

A method provided in accordance with aspects of the present disclosure includes determining a size of a target within a target plane, generating a plurality of quantum particles, converting the plurality of quantum particles into non-zero orbital angular momentum (OAM) quantum particles defining a wavefront phase vortex having a diameter corresponding to the size of the target, and directing the vortex to the target within the target plane.

In an aspect of the present disclosure, the vortex is configured to achieve perturbation while minimizing damage.

In an aspect of the present disclosure, converting the plurality of quantum particles to define the vortex having the corresponding diameter includes setting an L mode number (or quantum “charge”) of the vortex. Spurious perturbations are avoided or limited by adjusting the diameter of the vortex to a target (e.g., a cell or cell component) within the target plane.

In another aspect of the present disclosure, the target is a neuronal soma and the corresponding toroid diameter is greater than a nucleus of the neuronal soma and less than or equal to an overall size of the neuronal soma.

In another aspect of the present disclosure, directing the vortex to the target within the target plane includes determining a location of the target along the target plane and directing the vortex to the location of the target along the target plane.

In still another aspect of the present disclosure, multiple vortices are directed to multiple targets within one or more target planes.

In yet another aspect of the present disclosure, converting the plurality of quantum particles includes directing the plurality of quantum particles through a plurality of quantum particle manipulation components. Such components may include, for example, one or more optical components such as a physical or virtual lens configuration. A Spatial Light Modulator (SLM), an acousto-optical, and/or a Pockels Cell system may also be utilized. Other suitable devices for this purpose include deformable mirrors, adaptive (tunable) lenses, segmented waveplates, liquid crystal retarders, holographic diffractive optical elements, GRIN plates, and additively manufactured, photopolymerized, ablated or etched out surfaces.

In still yet another aspect of the present disclosure, the target is a neuron or sensor cell and the method further comprises perturbing the target with the vortex to activate opsins and induce action potential in the target. Additionally or alternatively, the method includes imaging the target. Such imaging may be performed by scanning the plural non-zero OAM quantum particles across the target and detecting the resulting effect such as one-or multi-photon excited fluorescence, luminescence, multiple harmonic generation, or Raman scattering (including any kind of coherent process).

In another aspect of the present disclosure, generating the plurality of quantum particles includes generating a plurality of photons. The plurality of photons may include, in aspects, two photons. In aspects, where the wavefront phase vortex is an optical vortex, converting the plurality of quantum particles may include converting the plurality of quantum particles into non-zero OAM quantum particles defining a wavefront phase optical vortex of super-twisted light.

### BRIEF DESCRIPTION OF THE DRAWINGS

Various aspects and features of the present disclosure are described hereinbelow with reference to the drawings wherein:

FIG. 1 is a simplified schematic of an optical system provided in accordance with the present disclosure;

FIG. 2 is a schematic illustration of a phase-only (SLM-based) process for generating non-zero orbital angular momentum (OAM) quantum particles in accordance with the present disclosure for perturbation and/or imaging;

FIG. 3 is a chart illustration of various features of different modes of toroidal, tow-photon excited intensities in a sample produced by non-zero OAM quantum particles (of varying “quantum charge,” “L”) in accordance with the present disclosure;

FIG. 4A is a cross-sectional image, in the Z-Y plane, of an optical toroid produced by non-zero OAM quantum particles in accordance with the present disclosure;

FIGS. 4B and 4C are cross-sectional images, in the X-Y plane, of an optical toroid produced by non-zero OAM quantum particles at various different positions in the X and Y directions, respectively, in accordance with the present disclosure;

FIG. 4D is a cross-sectional image, in the focal X-Y plane, of multiple, e.g., two, diagonally displaced in the X-Y direction, optical toroids produced by non-zero OAM quantum particles in accordance with the present disclosure;

FIGS. 5A and 5B illustrate experimental arrangements to mimic a target plane of cells of interest within a depth of tissue, e.g., for imaging or perturbation (FIG. 5A) or to detect the shape of light intensity produced in a thin dye layer (FIG. 5B);

FIGS. 6A and 6B are two-photon fluorescence images of GFP-expressing acute brain slices illustrating a pipette-patched cell under experimentation after 125 photostimulation events with optical vortices in accordance with the present disclosure prior to a Gaussian focused spot-only illumination event and after a Gaussian spot illumination event, respectively, (wherein damage from the Gaussian spot illumination event is circled in FIG. 6B for clarity); and

FIGS. 7A and 7B illustrate a phase pattern and resulting calculated near-field laser intensity distribution, respectively, of a super-twisted light optical vortex produced by non-zero OAM quantum particles with additional radial jump in phase and L-charge in accordance with the present disclosure.

### DETAILED DESCRIPTION

The present disclosure provides systems and methods for perturbation using plural non-zero orbital angular momentum (OAM) quantum particles. Although described hereinbelow with respect to two non-zero OAM photons used for optogenetic stimulation, it is understood that any other suitable non-zero OAM quantum particles may be utilized in accordance with the present disclosure, any other suitable number of plural non-zero OAM quantum particles may be utilized in accordance with the present disclosure, that other applications in addition or as an alternative to optogenetic stimulation are also contemplated such as, for example, imaging (e.g., by raster or circular scanning), and that other forms of perturbation other than stimulation are additionally or alternatively contemplated. The systems and methods of the present disclosure may find particular applicability, for example and without limitation, in brain-machine interfaces, optoacoustics (for readout), diagnostic microscopy or other microscopy or photodynamic therapy, light-based cochlear implants, and/or other body-machine interfaces and putative envisioned biological computing devices.

The use of plural non-zero OAM photons has been found to be advantageous in that the non-zero OAM carrying photons produce a toroid-shaped intensity profile in the near field, e.g., when focused by a lens, (also referred to as an optical waveform vortex) such that each non-zero OAM photon is delocalized in space and spans the whole toroid with zero electro-magnetic (EM) field amplitude at its center. The non-zero OAM of a photon is distinct from its polarization and, because photons can carry non-zero OAM, photons in this distinct quantum state are also referred to as “twisted light” and can have different scattering cross-sections compared to regular Gaussian and plane-wave photon beams of EM radiation. Additionally, the OAM of photons can be quantized to an integer “Laguerre-Gaussian mode” number, with this mode number determining the diameter and thickness of the toroid and the amount of angular momentum each photon holds. Thus, the optical vortex diameter can be adjusted according to the needs of the particular application. For example, the optical vortex diameter can be adjusted to match the size of a neuronal soma to allow for optogenetic stimulation of a large area of the soma membrane without damaging or minimizing damage to the central nuclear region of the soma.

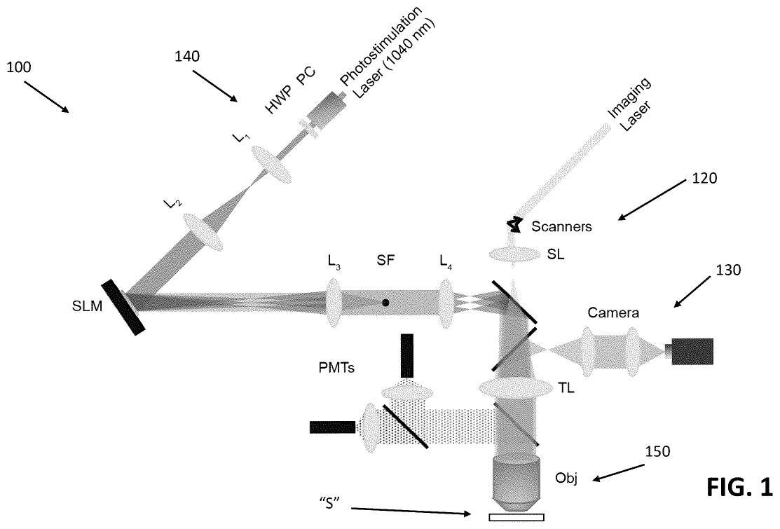

An optical system 100 provided in accordance with the present disclosure and configured for use in producing non-zero OAM photons for optogenetic stimulation to induce neuronal activity of a sample “S” and/or for imaging of neuronal activity of the sample “S” is described below with reference to FIG. 1.

Optical system 100 is shown in FIG. 1 including three distinct optical paths that are combined with one another: an imaging optical path 120, e.g., a laser-scanning two-photon fluorescence optical imaging path; a camera optical path 130, e.g., a wide-field epi-fluorescence optical camera path, and a photostimulation optical path 140, e.g., an SLM-based holographic two-photon photostimulation optical path. In aspects, one or more of these optical paths 120, 130, 140 may be omitted or combined, depending upon a particular purpose such as, for example, where one or more of imaging, camera-based verification, or stimulation is not utilized, or if optical vortices are used for imaging by raster scanning.

For two (or more) photon fluorescence imaging of neuronal activity, the imaging optical path 120 includes a tunable Ti:Sapphire femtosecond laser as a light source, e.g., operating at 920 nm. The beam passes through a Pockels Cell-based modulation system for power control and beam blanking during flyback and is expanded with a 1:3 beam expander lens pair before getting scanned with a resonant-galvo 6 mm mirror pair. The beam is expanded further by a scan-lens (50 mm) and tube-lens (200 mm) pair before the objective. Dual detection for the two-photon imaging is provided using a GaAsP or a multi-alkali PMT module pair (equipped with a High Voltage bias controller and a transimpedance current amplifier to generate measurable signal as output voltage) and a 735 nm long-pass dichroic, e.g., GFP/RFP filter set, was used to separate fluorescence into two color components. Other suitable configurations of the imaging optical path 120 are also contemplated.

To generate the non-zero OAM photons, the photostimulation optical path 140 includes another approximately 1040 nm fixed femtosecond laser with, in aspects, group delay dispersion (GDD) precompensation (e.g., Coherent Fidelity HP 10, 140 fs nominal pulse width). Downstream from this coherent light source is another Pockels Cell system for laser power control. Thereafter, the beam is expanded to fill a reflective phase-only Liquid Crystal on Silicon Spatial Light Modulator (SLM) and positioned to reflect light at a small angle (˜10 degrees) for digital hologram projection. To prevent interference from unmodulated light or ghost patterns, a virtual lens configuration is utilized, e.g., a virtual positive Fresnel lens phase pattern is computationally superimposed to the pre-calculated phase-mask displayed on the SLM via a Digital Interface. This focuses the modulated light at a different plane than the unmodulated (zero-order reflection) light to minimize spurious two-photon excitation. The L3 and L4 lens pair relays the SLM-generated phase pattern further towards the objective back aperture before the beam is directed into the microscope objective 150, e.g., a Movable Objective Microscope (MOM) microscope head, using a short-pass dichroic mirror situated past the scan lens but before the tube lens (to allow for overlap with the excitation imaging light). The imaging and photostimulation beams are focused onto the sample using, for example, a water-dipping 0.8 NA objective with a 3 mm working distance. Other suitable configurations of the photostimulation optical path 140 are also contemplated.

The camera optical path 130 enables verification of the location and shape of the optical vortices produced via the photostimulation optical path 140. The camera optical path 130 may include a long-pass dichroic mirror positioned between the scan lens and the tube lens, reflecting the excitation light to a 1:1 relay tube lens pair. The camera optical path 130 also includes an IR-blocking filter to ensure only visible fluorescence light is detected by the digital camera chip of the camera. Other suitable configurations of the camera optical path 130 are also contemplated.

Integration software for optical system 100 is utilized to apply an oscillating voltage to the Pockels Cell driver input, e.g., using an analog output controlled by graphical programming framework, since it has been determined that laser intensity modulation may increase the effectiveness of neuronal photostimulation via photostimulation optical path 140. Integration software for optical system 100 was also utilized to track and control the position of the objective 150 in X, Y, and Z dimensions, and to capture and display images from the digital camera. Further, integration software was utilized for X-Y adjustment of SLM phase lateral mask shifts to correct for misalignments in the photostimulation laser beam with respect to the center of the phase mask projected on the SLM. This adjustment enabled the production of uniform toroids of maximum average intensity along their perimeter.

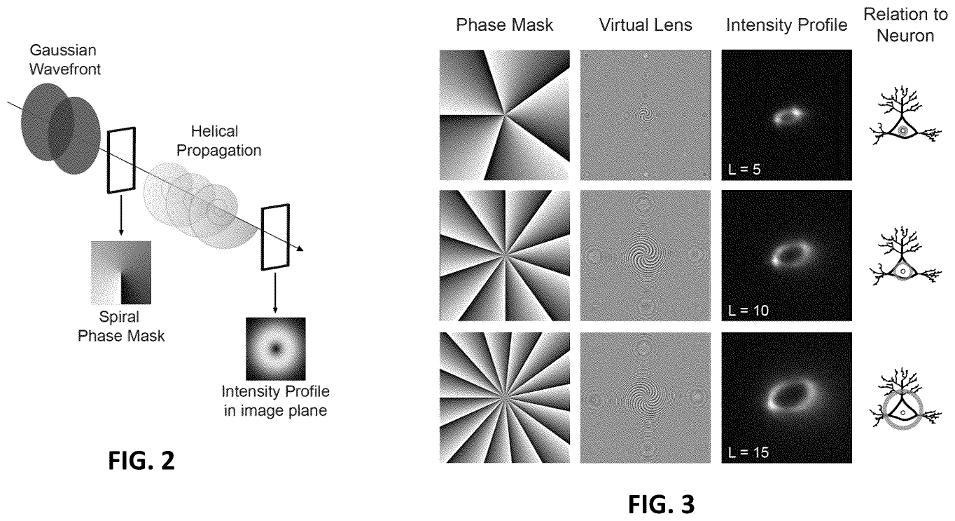

Turning to FIG. 2, the non-zero OAM photons generated by photostimulation optical path 140 of optical system 100 (see FIG. 1), as noted above, produce toroid-shape intensity profiles that are selectively adjustable to match the size of the target to be stimulated, e.g., a neuron's soma, thus enabling photostimulation of opsin molecules (simultaneously activating all opsins), in a neuron. The non-zero OAM quantum photons, more specifically, are generated by asymmetrically retarding a laser beam wavefront to induce a helical mode, e.g., using photostimulation optical path 140 of system 100 (FIG. 1). As illustrated schematically in FIG. 2, the transformation of a Gaussian beam into a non-zero OAM beam (by a spiral phase mask with vortex mode L=+1 or −1) generates a minimal-diameter toroid-shaped intensity field profile at the focal plane of the beam. Thus, the laser beam is transformed into a higher-order Laguerre-Gaussian mode that can be viewed as a composition of photons carrying non-zero OAM, thereby providing the plural non-zero OAM photons.

With additional reference to FIG. 3, various different SLM diffraction patterns for the SLM of the photostimulation optical path 140 of optical system 100 (see FIG. 1) are shown in the column titled “Phase Mask,” corresponding to different vortex modes: L=+/−5, L=+/−10, and L=+/−15, respectively. Of course, these modes are exemplary, and any suitable vortex mode may be utilized depending upon a particular purpose. Further, it is noted that, the virtual lens configuration detailed above with respect to photostimulation optical path 140 of optical system 100 (see FIG. 1) and shown in the column titles “Virtual Lens” for each of the vortex modes in FIG. 3 functions to remove unwanted laser speckle pattern and/or bright center spot originating from direct reflection off the SLM surface while maintaining the ability of the optical vortex in the object plane to be detected with the digital camera. The measured lateral (XY) intensity profile of the non-zero OAM beam in each of the vortex modes is shown in the column titled “Intensity Profile” in FIG. 3.

Continuing with reference to FIG. 3, and as noted above, the diameter of the toroid can be adjusted, e.g., to tune the OAM photons to the size of the soma, by adjusting the vortex mode. The exemplary vortex modes illustrated in FIGS. 3 (L=5+/−, L=1+/−0, and L=+/−15) were chosen to match the typical size of a neuron's soma, although, as noted above, any suitable vortex mode may be utilized depending upon a particular purpose. More specifically, as shown schematically in the column titled “Relation to Neuron” in FIG. 3, the diameter of the toroid in each of the three vortex modes is shown in relation to a typical neuron, thus indicating the tunability of the OAM photons to facilitate photostimulation. In aspects, sizing the vortex such that the vortex diameter is larger than the nucleus and substantially equal to or smaller than the soma, e.g., as shown in the L=+/−10 image of the column titled “Relation to Neuron” in FIG. 3, facilitates effective and efficient stimulation without cell damage.

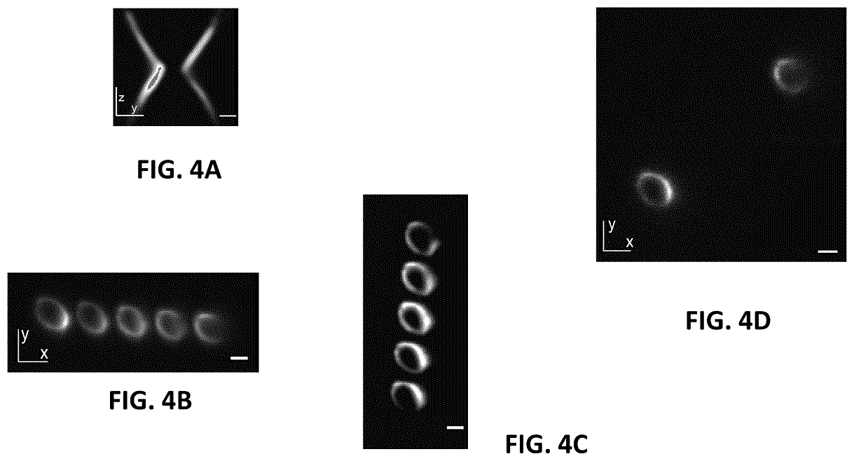

Turning to FIGS. 4A-4D, with respect to the target or focal plane, the optical vortices are focused and controlled such that the non-zero OAM photons generate fluorescence at or near the focal plane, with intensity rapidly vanishing 15-20 μm away from the focal plane. This is illustrated, for example, in the two-photon fluorescence intensity profile generated by an optical vortex with mode L=+/−15 shown in FIG. 4A.

The location of the optical vortex can also be moved within the target plane, by recalculation of the SLM phase mask, while preserving the shape of the optical vortex, although the intensity profile may vary slightly with position, as illustrated in FIGS. 4B and 4C which show successive positions of the optical vortex in the X and Y directions, respectively.

In addition, multiple vortices can be generated simultaneously for targeting multiple spatially dispersed neurons (or other suitable structures), as illustrated in FIG. 4D. Although two vortices are shown for illustration purposes, any suitable number of vortices can be generated.

Thus, as detailed above, the optical vortex produced by the non-zero OAM photons can be substantially limited to the target plane (see FIG. 4A), can be resized (e.g., by varying the toroid diameter) (see FIG. 3), and can be moved within the target plane (see FIGS. 4B and 4C). In addition, multiple optical vortices can be produced at the target plane (see FIG. 4D). Accordingly, tuning of the size, location in plane, location along optical axis (Z) using Virtual Lens power adjustment, and/or number optical vortices can be readily achieved to stimulate a particular structure or structures within reasonable bounds in all 3 dimensions of space.

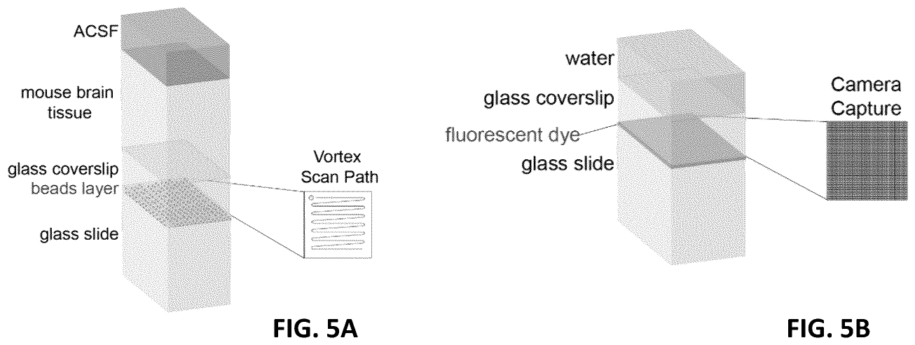

With respect to tissue depth considerations, it was experimentally confirmed the non-zero OAM photons of the present disclosure are capable of penetrating through tissue depths, e.g., of mouse brain tissue, of at least 200 μm and, in aspects, to depths of at least 2-3 mm. Further, it was observed that the non-zero OAM photons are sufficiently unaffected by to variations of refractive index in biological tissue and do not generate spurious focal regions, which may cause tissue damage. FIGS. 5A and 5B illustrate experimental setups to mimic a target plane of cells of interest within a depth of tissue. The non-zero OAM photons achieved the toroidal shape at the target planes in these experiments and, thus, were sufficiently insensitive to variations of refractive index in biological tissue such that the non-zero OAM photons did not generate spurious focal regions that could potentially damage tissue.

In aspects, in order to tune the system 100 (FIG. 1) to facilitate activating opsins and induce action potentials in neurons, in addition to the sizing and positioning of the non-zero OAM photons detailed above, the laser power can be controlled using different Pockels Cell control voltages. Indeed, electrophysiological responses (e.g., action potentials) were achieved using exposure-controlled photostimulation with 1040 nm ultrafast pulsed laser light, sinusoidally or otherwise modulated by a Pockels Cell, for all target cells under experimentation with a low timing jitter and low latency. Further, the non-zero OAM photons did not notably damage cells in this experimentation, even when maximum, continuous photostimulation power was applied.

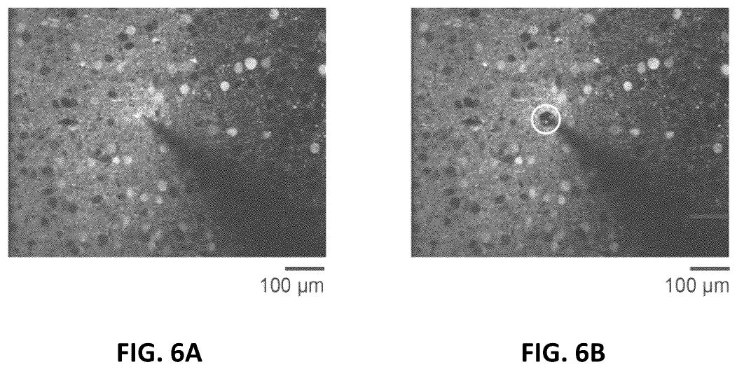

Turning to FIGS. 6A and 6B, as noted above, the aspects and features of the present disclosure enable stimulation without tissue or cell photodamage. Experimental results illustrating this are shown in FIGS. 6A and 6B. More specifically, FIG. 6A illustrates a two-photon fluorescence image of a cell after 125 photostimulation events in accordance with the above-detailed aspects and features of the present disclosure. The photostimulations were performed using an average power of approximately 250 mW with modulation. Notably, minimal or no cell damage is identified. FIG. 6B, on the other hand, shows the same cell, but after a single Gaussian focused spot illumination event at the same exposure time, power, and modulation. As highlighted in FIG. 6B, brain tissue damage in the form of an ablation hole was created in the place of a former viable cell as a result of the Gaussian spot illumination event.

Other applications for the systems and methods of the present disclosure include: photoperturbation or photomanipulation, e.g., optically or thermally disrupting, inhibiting, and/or activating, intracellular structures such as organelles and proteins, and/or intracellular processes, with a vortex of plural non-zero OAM photons; measuring the microviscosity, or liquid-liquid phase separation with a vortex of plural non-zero OAM photons to detect and/or disrupt anomalies and, in aspects, alter the phase concentration in particular regions of a cell or group of cells; detecting and/or treating intracellular anomalies or diseases with a vortex of plural non-zero OAM photons such as, for example, by mechanically disrupting, activating, and/or changing the direction of mitochondria to detect and/or treat mitochondrial disease; and detecting and/or treating lysosomal storage diseases with a vortex of plural non-zero OAM photons. Another application may include cochlear implants whereby optical encoding of sensory signals is used to transmit information to neuronal fibers with minimal photodamage to the live cellular structures involved due to application of the non-zero OAM photons as disclosed hereinabove.

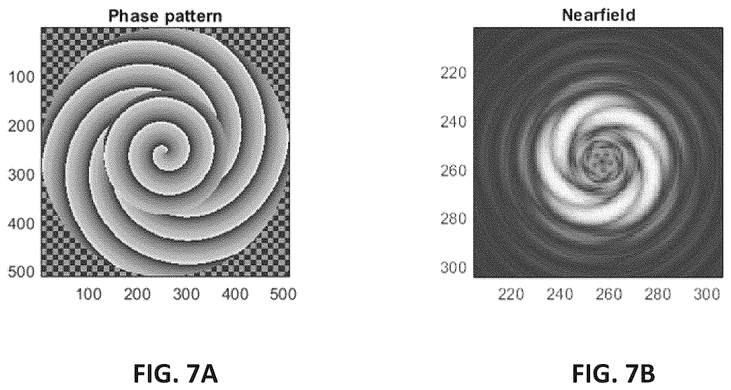

With reference to FIGS. 7A and 7B, in aspects, the optical vortex produced by the non-zero OAM photons can define different configurations such as, for example, by using twisted light to create a super-twisted light optical vortex, e.g., a twisted toroid. Super-twisted light is characterized by its corkscrew-shaped wavefronts having twist lobes that can be controlled in number, intensity, and/or direction. More specifically, with respect to cell body photostimulation for example, the direction (sign) of twist and/or light polarization state can be chosen in accordance with the cell structure(s) to be stimulated to facilitate stimulation, as cells are made of asymmetrical molecules. Likewise in other applications, super-twisted light can be controlled to customize the optical vortex for the particular purpose utilized.

Also contemplated for use in accordance with the present disclosure are quantum mechanically entangled photons. That is, in aspects, two or more of the plurality of non-zero OAM photons defining the optical vortex may be entangled in the sense of being created as two or more indistinguishable quantum particles. This can be used for “ghost” imaging, remote sensing, and/or other applications where such quantum mechanical phenomena are beneficial.

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The aspects described with reference to the attached drawings are presented only to demonstrate certain examples of the disclosure. Other elements, steps, methods, and techniques that are insubstantially different from those described above and/or in the appended claims are also intended to be within the scope of the disclosure.

What is claimed is:

1. A method, comprising:
determining a size of a target within a target plane;
generating a plurality of quantum particles;
converting the plurality of quantum particles into non-zero orbital angular momentum (OAM) quantum particles defining a wavefront phase vortex having a diameter corresponding to the size of the target; and
directing the vortex to the target within the target plane.

2. The method according to claim 1, wherein directing the vortex to the target includes perturbating the target while minimizing photodamage.

3. The method according to claim 1, wherein converting the plurality of quantum particles to define the vortex having the corresponding diameter includes setting an L mode number of the vortex.

4. The method according to claim 1, wherein the target is a neuronal soma and wherein the corresponding diameter is greater than a nucleus of the neuronal soma and less than or equal to an overall size of the neuronal soma.

5. The method according to claim 1, wherein the vortex is configured to limit or avoid spurious stimulations when the diameter of the vortex corresponds to the size of the target.

6. The method according to claim 1, wherein directing the vortex to the target within the target plane includes determining a location of the target within the target plane and directing the vortex to the location of the target within the target plane.

7. The method according to claim 1, wherein multiple vortices are directed to multiple targets within the target plane or multiple target planes.

8. The method according to claim 1, wherein converting the plurality of quantum particles includes directing the plurality of quantum particles through one or more quantum particle manipulation components.

9. The method according to claim 1, wherein the target is a neuron and wherein the method further comprises perturbating the target neuron with the vortex to activate opsins and modify action potential in the neuron.

10. The method according to claim 1, further comprising imaging the target using the vortex.

11. The method according to claim 1, wherein each quantum particle of the plurality of quantum particles is a photon.

12. The method according to claim 1, wherein the plurality of photons includes two photons.

13. The method according to claim 1, wherein the vortex includes super-twisted light.

14. A system, comprising:
a source of quantum particles configured to generate a plurality of quantum particles;
a plurality of quantum particle manipulation components configured to convert the plurality of quantum particles into non-zero orbital angular momentum (OAM) quantum particles defining a wavefront phase vortex; and
an objective or focusing lens configured to direct the vortex to a target plane within a depth of tissue.

15. The system according to claim 14, further comprising a mode selector configured to select a mode of the vortex to adjust at least one parameter of the vortex at the target plane.

16. The system according to claim 15, wherein the mode selector is configured to adjust a diameter of the vortex at the target plane to correspond to a size of a target.

17. The system according to claim 15, wherein the vortex is configured to limit or avoid spurious stimulations when the mode is selected such that a diameter of the vortex substantially match a size of a target at the target plane.

18. The system according to claim 14, further comprising an adjustment mechanism configured to adjust a location of the vortex within the target plane.

19. The system according to claim 18, wherein the adjustment mechanism is configured to adjust the location of the vortex within the target plane to correspond to a location of a neuron.

20. The system according to claim 14, further comprising a multiplier mechanism configured to generate multiple wavefront phase vortices in one or more target planes.

21. The system according to claim 20, wherein each vortex of the multiple vortices corresponds to a different neuron.

22. The system according to claim 14, wherein the plurality of quantum particle manipulation components includes at least one of: a physical lens configuration, a virtual lens configuration, a Spatial Light Modulator (SLM), an acousto-optical system, a Pockels Cell system, a deformable mirror, a tunable lens, a segmented waveplate, a liquid crystal retarder, a holographic diffractive optical element, a GRIN plate, an additively manufactured surface, a photopolymerized surface, an ablated surface, or an etched surfaces.

23. The system according to claim 14, wherein each quantum particle of the plurality of quantum particles is a photon.

24. The system according to claim 23, wherein the plurality of photons includes two photons.

25. The system according to claim 14, wherein the vortex is configured to achieve perturbation while minimizing live cell damage.

26. The system according to claim 14, wherein the vortex includes super-twisted light.
